(19) United States
(12) Patent Application Publication
(10) Pub. No.: US 2002/0188181 A1
(43) Pub. Date: Dec. 12, 2002

(54) Interface for a portable point-of-care patient diagnostic device

(76) Inventors: Christopher S. Boit, Wellesley, MA (US); Michael K. Dempsey, Westford, MA (US); William M. Reed, Carlisle, MA (US)

Correspondence Address: PHILIPS ELECTRONICS NORTH AMERICAN, 580 WHITE PLAINS ROAD, TARRYTOWN, NY, 10591, US

(21) Appl. No.: 09/876,692

(22) Filed: Jun. 7, 2001

(51) Int. Cl. A61B 5/00

(52) U.S. Cl. 600/300

(57) ABSTRACT

An interface or base station is provided for a portable point-of-care patient monitoring and diagnostic device (PCP device) to provide at least power input to the PCP device to extend the battery life thereof, to facilitate data transfer with the PCP device in both directions, to provide an enhanced data input capability for the PCP device and to provide a data output/display capability for the PCP device. At least a portion of the base station may itself be portable for enhanced patient mobility.

### FIELD OF THE INVENTION

This invention relates to portable point-of-care patient monitoring and diagnostic devices, and more particularly to a portable mechanical and electrical interface for such devices which provides at least power input, data transfer, data input and data output/display capabilities.

### BACKGROUND OF THE INVENTION

Portable bedside/point-of-care patient diagnostic and monitoring devices (PCP devices) are currently used extensively in hospital and other facilities providing medical care to relatively inexpensively monitor and/or provide diagnostic information concerning a patient, while permitting the patient to be ambulatory within a prescribed area. Such devices typically include a physiological monitor, for example a heart monitor, noninvasive blood pressure measuring device, a saturated oxygen monitor or various other point-of-care testing devices. Such devices may also include a processor, which is generally a suitably programmed microprocessor chip or special purpose processor performing very limited functions on information received from the processor and sending results of such processing, either periodically or as requested/required, for example when interrogated, to a transmitter/receiver which forwards the information to a processor at a central station, for example a processor for a ward or unit or a central processor for the hospital or other institution. These devices may also receive limited inputs from the central station processor to the transmit/receive unit, for example interrogation inputs or inputs causing minor programming changes to the device processor. Such devices may also have an I/O port, either in addition to or instead of the transmit/receive unit, through which the device may either output its information, either as produced by its monitor or as processed by its processor, and/or receive interrogation, reprogramming or other appropriate inputs. Finally, such devices typically have a battery for providing power to the physiological monitor, processor, transmit/receive unit and/or I/O port.

While such devices are useful, they also have a number of limitations. First, in order to keep the cost of such devices as low as possible, they typically have either no capability for providing inputs at the device, or only a very limited such capability, for example a panic button which a patient can press when in distress. Similarly, they typically do not have a local display or other mechanism for permitting a caregiver to obtain information from the device itself, the caregiver being required to either run to the central station to obtain such information if required or to communicate with the central station by telephone, walky talky or the like, in order to determine physiological readings from the device. This is a nuisance when a nurse or other caregiver is doing rounds and the caregiver either needs to fill in the information from the monitor after rounds are over, run to the central station to obtain such information in the middle of rounds, or place a call to what can frequently be a busy central station in an effort to obtain the information. It is even a bigger problem during a medical emergency when the caregiver does not have easy access to the monitor outputs, information which may be critical in dealing with the emergency. It is also a problem when the device is being mounted to the patient and there is a need to assure that the device is both mounted at the right location and that good contact has been made. With current devices, this information is not readily available to the caregiver doing the mounting. Similarly, during the administering of certain drugs or the performance of other medical procedures, there is sometimes a need to monitor the effect of such drug or procedure on a monitored physiological condition of the patient, something which is not easily accomplished with current PCP devices.

The lack of input capability is also a problem in that a caregiver, for example on rounds, cannot input information to the device for transmission through the device telemetry unit to the central station. Further, since it is desirable that such devices be electrically isolated from other devices to protect the patient, to the extent such devices have an I/O port, it is typically an optical port, requiring the caregiver to carry an optical cable in order to communicate with the device and also that the caregiver carry some type of external I/O device, for example a handheld or laptop computer, to permit I/O functions to be performed through the optical cable. This can be a burden on the caregiver in that it requires the caregiver to carry additional equipment when on rounds or when responding to a medical emergency, and in that time is required to establish the optical connection between the I/O device and the PCP device, making the caregiver less efficient on rounds and taking up precious time in the event of a medical emergency. It would therefore be preferable if an I/O capability could be established with PCP devices quickly and easily, and preferably with a device inexpensive enough so that it can stay with the patient in a variety of situations, including patient transport situations.

Finally, since the PCP devices are battery operated, they have a limited period of operation before battery replacement is required, and it is important that batteries be replaced frequently enough so that battery failure on a device does not occur, since this could result in the patient being unmonitored during a medical emergency, preventing timely detection of such emergency. Since battery replacement is performed by caregivers, frequent battery replacement can be expensive in terms of both time and material, and it would be preferable if the life of the batteries could be extended as long as possible without jeopardizing patient safety.

Therefore, while existing PCP devices serve a useful function, there is a need to supplement such devices in a manner so as to provide enhanced battery life and easier input/output capability at the patient site.

### SUMMARY OF THE INVENTION

In accordance with the above, this invention provides a base station or interface for use with a point-of-care patient (PCP) device of the type having at least one physiological monitor, an I/O port through which information from the monitor may be passed, a battery, and a power port. The base station includes a housing having a slot formed therein which is sized and shaped to permit the PCP device to seat therein with only a single orientation; a station I/O port positioned in the slot so as to interface with the PCP device I/O port when the PCP device is fully seated in the slot and; a station power port positioned in the slot so as to electrically connect with the PCP device power port when the PCP device is fully seated in the slot, electric power being at least selectively applied to the station power port. The station I/O port may be connected to a processor for receiving and utilizing information from the PCP monitor(s). The source of electrical power and the processor may be remote from the remainder of the base station and may be connected thereto by suitable wiring or other suitable link, or the station may include the processor and may also include an output mechanism and an input mechanism, the processor receiving information from the PCP device, controlling outputs on its output mechanism and providing inputs to control the PCP monitor(s) under control at least in part of the station input mechanism. The slot preferably includes a latch which engages the PCP device when it is fully seated in the slot, the latch preferably being a “soft latch,” providing some resistance to removal of the PCP device from the slot, but not significantly inhibiting such removal. For preferred embodiments, the I/O ports are both optical ports, and the power ports are either galvanic connections, inductive couplings or RF connections. The base station preferably includes a battery for operating this station at least when the station has a PCP device mounted in its slot and is being moved, thereby rendering the base station completely portable.

The invention also includes a patient monitoring system which includes both a PCP device and a portable base station of the types indicated above.

The foregoing and other objects, features and advantages of the invention will be apparent from the following more particular description of preferred embodiments of the invention as illustrated in the accompanying drawings.

### IN THE DRAWINGS

FIG. 1 is a top front perspective view of a portable base station in accordance with an illustrative embodiment of the invention with a PCP device about to be mounted therein;

FIG. 2 is a front sectional view of the portable base station shown in FIG. 1 and of the PCP device about to be mounted therein;

FIGS. 3A and 3B are a front view and a right side view, respectively, of the portable base station shown in FIG. 1 with the PCP device mounted therein;

FIG. 4 is a semi-schematic block diagram of a portable base station with a PCP device mounted therein for an illustrative embodiment; and

FIG. 5 is a front view of a portable base station having a PCP device partially mounted therein for an alternative embodiment of the invention where the PCP receiving slot is separate from the remainder of the portable base station, being connected thereto by a suitable cable.

### DETAILED DESCRIPTION

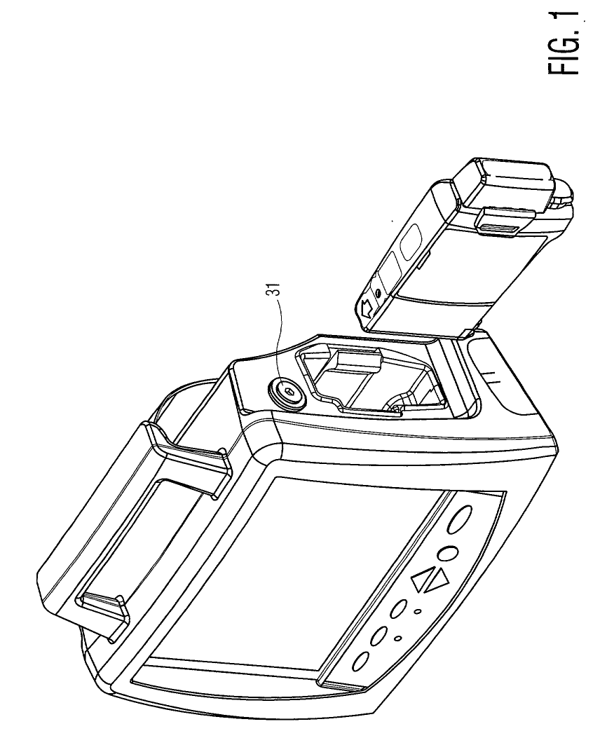

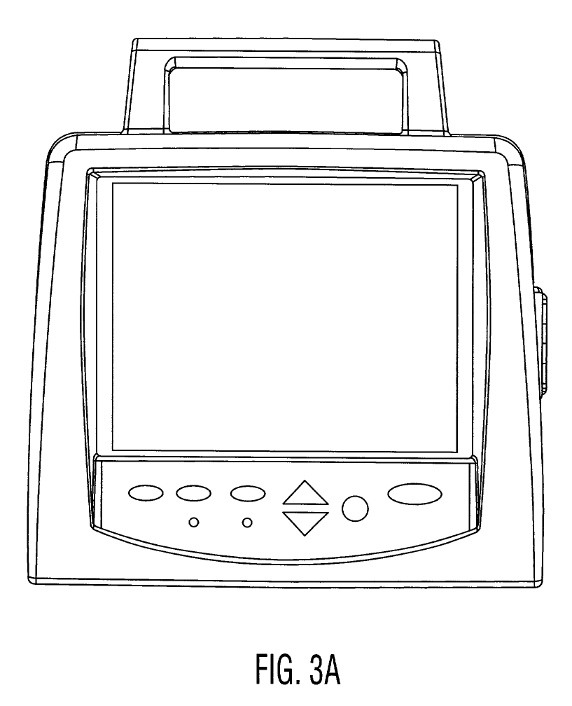

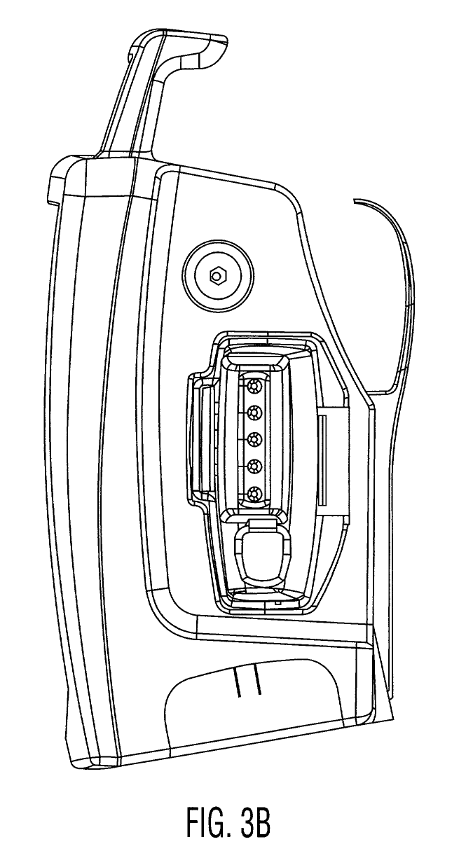

Referring to FIGS. 1, 2 and 3, the patient monitoring system 10 of this invention includes a portable base station 12 having a slot 14 formed therein for receiving a point-of-care patient (PCP) device 16. As may be best seen, for example in FIG. 3B, PCP device 16 has an asymmetric cross-section, and slot 14 has a corresponding cross-section so that PCP device 16 fits into slot 14 with only a single possible orientation. As may be best seen in FIGS. 1 and 3B, a flexible latch 18 is either formed or mounted in the back wall of slot 14, the leading side of latch 18 having a projection 20 with a substantially triangular cross-section, the two extending walls of projection each being at approximately a 45° angle for a preferred embodiment. The tension on latch 18 is relatively light so that when PCP device 16 is inserted in slot 14, projection 20 is easily cammed out of the way of the device by the leading edge thereof and drops over the rear of shoulder 22 on PCP device 16 when the device is fully seated in slot 14 to provide a tactile, visual, and audio indication to an operator that the PCP device is properly seated in the slot and to inhibit spurious removal of the PCP device from the slot. However, latch 18 is a “soft latch,” the tension on the latch not being sufficient to significantly impede removal of the PCP device from the slot by an operator when desired, the angle of the rear face of head 20 permitting the head to easily ride up over shoulder 22 when a removal force is applied to device 16.

Station 12 has a display screen 24 which is preferably a flat screen display such as a standard liquid crystal display, light-emitting diode array, or the like. The device also has an array of keys or buttons 26 which may be utilized for inputting information to the device. While a full alphanumeric keyboard may be provided, and is within the contemplation of the invention, for the illustrative embodiment shown, the display is normally a menu which may be incremented, decremented, selected, and otherwise utilized by operating selected keys in ways known in the art. Further, while a keyboard 26 is shown as the input mechanism for the illustrative embodiment shown in the Figures, this is not a limitation on the invention, and other suitable input devices, for example voice activation, may also be utilized. Since station 12 is to be portable, the station also has a handle 28 for carrying the station. For the illustrative embodiment, a holder 30 is also provided on the back of the device for storing cables or wires, for example, patient leads, when not in use. Holder 30 may for example be flexible, permitting cables/leads of varying size to be stuffed behind the holder which resiliently bends to permit the cables/leads to be inserted and to then hold them in place. A socket 31 is provided above slot 14 which may for example be used for a blood pressure connection and other suitable connectors may also be provided as required.

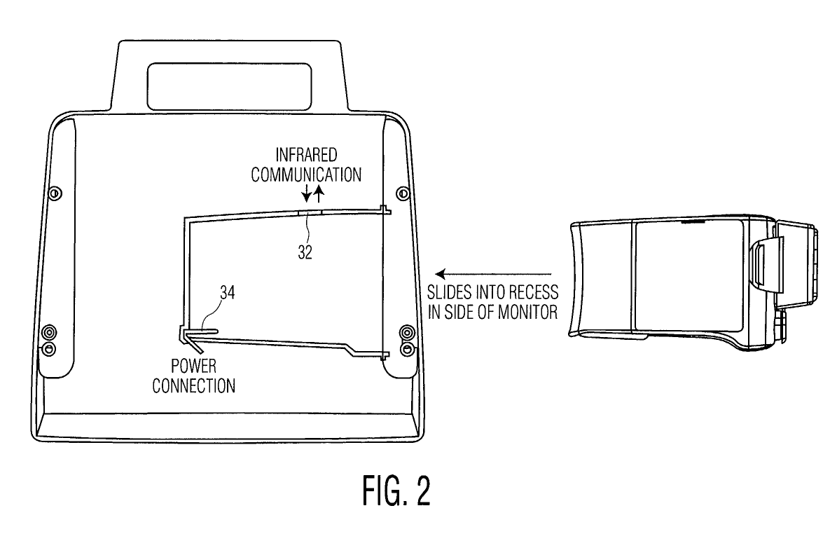

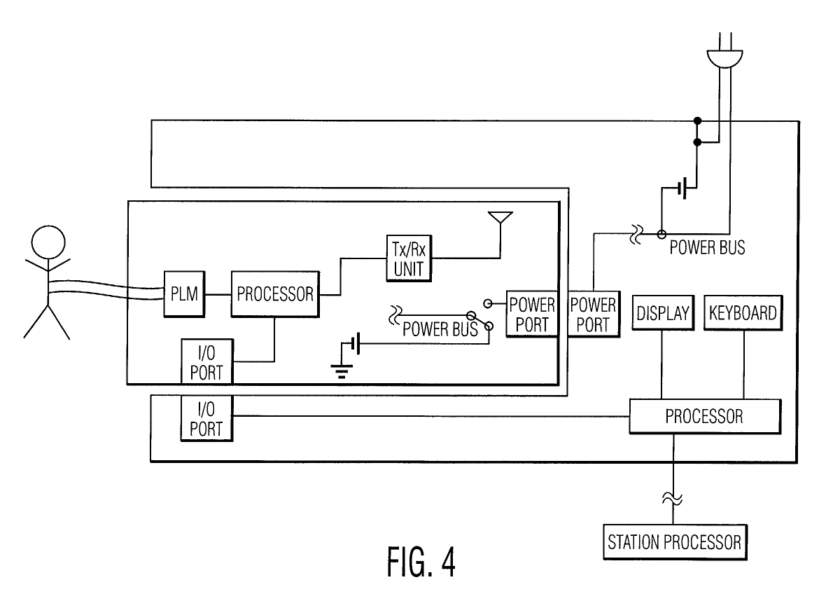

Referring more specifically to FIGS. 2 and 4, it is seen that in addition to latch 18, slot 14 also contains a communications port 32 and a power connection port 34. For preferred embodiments, communications port 32 is an optical port, for example an infrared communications port, which communicates with a corresponding optical port 36 on PCP device 16. However, while ports 32 and 36 are infrared or optical ports for preferred embodiments primarily because this is the preferred way of communicating with the existing PCP device being interfaced to, the use of such ports is not a limitation on the invention. Thus, this port could also be a galvanic port, an RF port, or some other type of communications port either currently used or hereafter developed. Similarly, power port 34 is shown at the rear of slot 14 as a galvanic connection which mates with a corresponding terminal or port 38 on device 16. However, this again is not a limitation on the invention, and power transfer may be accomplished in other ways known in the art including, but not limited to, inductive couplings and RF connections. The important thing is that station 12 is capable of easily providing both a communications and a power connection to PCP device 16 by merely mounting the device in slot 14.

FIG. 4 is a schematic representation of illustrative circuitry for both PCP device 16 and portable base station 12, the Figure showing PCP device 16 mounted in slot 14 of base station 12. PCP device 16 includes a physiological monitor 40 which may for example be a heart monitor, a noninvasive blood pressure monitor, a saturated oxygen monitor, or other suitable point-of-care testing device. Monitor 40 is connected by suitable cables 42 to the patient 44. Monitor 40 is also connected to a small processor 46 which is programmed to receive outputs from monitor 40 and to perform some processing on such outputs to place them in better form for outputting, for example performing analog-to-digital conversion, data compression, conversion to a form more easily outputted by the output mechanism utilized, or the like. Processor 46 may also provide inputs to monitor 40 to for example trigger the taking of readings, trigger the outputting of readings, control ranges on the monitor, or perform other control functions on monitor 40, as required. Processor 46 receives inputs from and outputs data to I/O port 36 and also receives data from and transmits data to transmit/receive (T/R) unit 48 which is connected to antenna 50. TR unit 48 and antenna 50 may be utilized to transmit data to and receive data from station processor or a facility central processor (not shown), particularly when PCP device 16 is not mounted in base station 12. However, T/R unit 48 may also be used, as discussed in greater detail later, when PCP device 16 is mounted in base station 12.

As indicated earlier, I/O port 36 is utilized to transmit data from processor 46 to I/O port 32 of base station 12 or to receive data from the base station through I/O port 32. Proper seating of PCP device 16 in slot 14 assures that I/O ports 32 and 36 are properly aligned to facilitate such data transfer.

Finally, device 16 has a power port 38 which is aligned with station power port 34 to receive power from base station 12 when device 16 is properly seated in slot 14, and a battery 54. Battery 54 is normally connected through a switch 56 to a power bus 58 of device 16, the power bus being connected to drive I/O port 36, monitor 40, processor 48, and T/R unit 48. When power ports 34 and 38 are aligned so that power from base station 12 is being applied to power port 38, switch 56 may be mechanically, magnetically or otherwise transferred to terminal 60 so that bus 58 is receiving power from base station 12 rather than from battery 54, thereby reducing the drain on battery 54 and extending its life. Alternatively, both terminal 60 and terminal 62 of switch 56 may be connected to power bus 58 so that the bus is obtaining power in parallel from both sources and/or battery 54 is recharged from device 12 through power port 38 while power is being applied to the power bus from base station 12. In either event, the life of battery 54 is extended by mounting device 16 in base station 12, permitting PCP devices 16 to operate for significantly longer periods of time without maintenance for battery testing and change.

Base station 12 also has a processor 64 which is preferably a larger and more versatile processor than processor 46, and which can therefore be programmed to perform a significantly greater number of functions than processor 46. Processor 64 is connected to control the output appearing on display 24 or on another suitable output device for base station 12, and is also connected to receive inputs from keyboard 26 and, to the extent necessary, to provide control outputs thereto. Finally, processor 64 is connected to receive inputs from and to provide outputs to I/O port 32, and may also be connected through a bus 66 to station processor 52, which may for example be located at a nursing station in the facility or to the facility central processor. Base station 12 also has a line cord 68 terminating in a plug 70 which may be connected to a standard wall outlet, and an optional battery 72 which is preferably a larger, more powerful battery than battery 54 of PCP device 16. Battery 72 may for example be mounted behind door 73 (FIG. 1). When current is not being applied to line 68, power bus 74 receives current from battery 72, power bus 74 being connected to drive display 24, keyboard 26, ports 32 and 34, and processor 64. When plug 70 is plugged in, current from line 68 is applied to power bus 74, either in parallel with battery 72, or the line current on bus 74 may also be used to charge battery 72. Power bus is also connected to power port 34 to provide power to device 16, as indicated earlier. While in order to maximize portability of base station 12 and to assure operation thereof in the event of a power failure, the inclusion of a battery 72 is clearly preferred, the base station can also operate only from line cord 68, with battery 72 being eliminated.

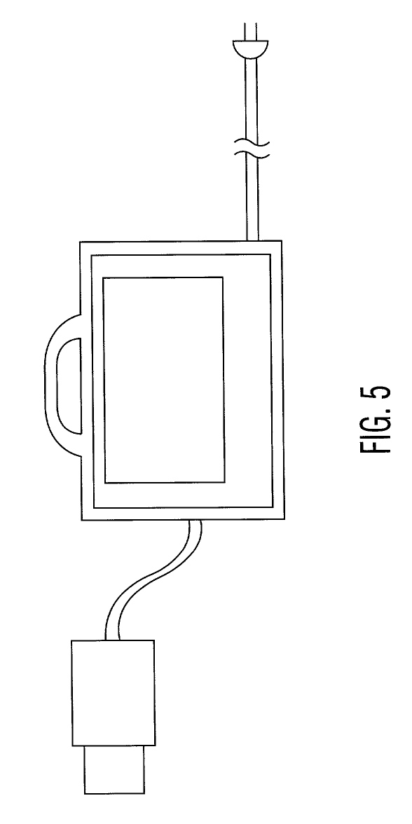

FIG. 5 illustrates an alternative embodiment of the invention wherein base station 12 is divided into a portion 12A which includes slot 14 and ports 32, 34 and a main portion 12B which contains display 24, keyboard 26, processor 64, line cord 68 and battery 72. Portions 12A and 12B of the base station are interconnected by a suitable cable 76. The embodiment of FIG. 5 is useful in applications where space at the point-of-care, for example at the patient's bed, wheelchair or gurney, is limited, and it is preferable that most of base station 12 be located slightly spaced from the point-of-care for easier viewing and manipulation by the caregiver and to afford easier access to the patient, while still having docking slot 14 close to the patient to facilitate mounting and removal of the PCP device. The embodiment of FIG. 5 also isolates processor 64 and other potentially radiation generating components of device 12 from other equipment at the point-of-care to minimize potential interference between such devices. Patient mobility may be enhanced by linking portions 12A and 12B by an RF or other suitable telemetry device, rather than by cable 76. Except as indicated above, the embodiment of FIG. 5 operates in the same manner as the embodiment of FIGS. 1-4, and what has been said for this earlier embodiment, applies equally with respect to the embodiment of FIG. 5.

A patient monitoring system has thus been provided which simply and inexpensively supplements existing PCP devices to provide point-of-care input and output capabilities so that a caregiver at the point-of-care can more easily attach the PCP device to the patient, can interrogate the PCP device to obtain desired information, and can quickly and easily view information obtained by the PCP device at the point-of-care, for example during rounds or in an emergency situation, thereby facilitating better patient care and more efficient usage of the caregiver's time. Base station 12 may be located at the point-of-care, or the caregiver, who is for example on rounds, may carry base station 12 with him to facilitate I/O from the PCP units. The use of device 12 also supplements and/or recharges battery 54 of the PCP device to significantly extend battery life of such devices, thus extended the time between required maintenance for testing and replacement of the batteries. Base station 12 also permits a caregiver to input information at the point-of-care which may either be outputted through bus 66 to a station or central processor, or which may be sent through I/O ports 32 and 36, and processor 46 to be outputted by T/R unit 48 through antenna 50. Finally, since base station 12 is also battery operable, the station may be unplugged and remain with the patient when the patient is being transported, for example on a gurney to surgery, to permit continued visual monitoring by the caregiver of a patient's condition when the patient is in crisis situation, or to permit a very sick patient to leave bedside monitors and move about the facility, for example in a wheelchair, while still permitting either the patient himself or a caregiver to easily monitor the patient's condition and/or to more easily respond to a patient emergency.

While the invention has been particularly shown and described above with respect to preferred embodiments, and various modifications in selected components have been discussed throughout the specification, other modifications are also possible. For example, processor 46, and possibly processor 64, rather than being programmable devices, could be special purpose hardware or hybrid devices for performing limited functions. In suitable applications, T/R unit may not be present and/or bus 66 may not be utilized. Thus, while the invention has been particularly shown and described above with reference to preferred embodiments, the foregoing and other changes in form and detail may be made therein by one skilled in the art while still remaining within the spirit and scope of the invention which is to be defined only by the appended claims.

What is claimed is:

1. A base station for a point-of-care patient (PCP) device of a type having at least one physiological monitor, an I/O port through which information from said monitor may be passed, a battery and a power port, the base station including:
a housing having a slot formed therein, which slot is sized and shaped to permit said PCP device to seat therein with only a single orientation;
a station I/O port positioned in said slot so as to interface with said PCP device I/O port when said PCP device is fully seated in said slot; and
a station power port positioned in said slot so as to electrically interconnect to said PCP device power port when said PCP device is fully seated in said slot, electric power being at least selectively applied to said station power port.

2. A base station as claimed in claim 1 wherein said station I/O port is connected to a processor for receiving and utilizing information from said at least one monitor.

3. A base station as claimed in claim 2 wherein a source of electrical power and said processor are remote from said base station and are connected thereto by a suitable link.

4. A base station as claimed in claim 2 wherein said station includes said processor, an output mechanism and an input mechanism, said processor receiving information from said PCP device, controlling outputs on said output mechanism, and providing inputs to control said at least one monitor under control at least in part of said input mechanism.

5. A base station as claimed in claim 1 wherein said slot includes a latch which engages when said PCP device is fully seated in said slot.

6. A base station as claimed in claim 5 wherein said latch is a soft latch, providing some resistance to removal of the PCP device from the slot, but not significantly inhibiting such removal.

7. A base station as claimed in claim 1 wherein said 1/0 ports are both optical ports.

8. A base station as claimed in claim 1 wherein said power ports are one of galvanic connections, inductive couplings, and RF connections.

9. A base station as claimed in claim 1 including a battery for operating said station at least when said station has a PCP device mounted in its slot and is being moved, said base station thus being portable.

10. A patient monitoring system including:
a point-of-care patient (PCP) device of a type having at least one physiological monitor, an I/O port through which information from said monitor may be passed, a battery and a power port; and;
a base station including a housing having a slot formed therein, which slot is sized and shaped to permit said PCP device to seat therein with only a single orientation; a station I/O port positioned in said slot so as to interface with said PCP device I/O port when said PCP device is fully seated in said slot; and a station power port positioned in said slot so as to electrically connect to said PCP device power port when said PCP device is fully seated in said slot, electric power being at least selectively applied to said station power port.

11. A system as claimed in claim 10 wherein said battery is connected to be charged by electric power applied to the PCP device through said power ports.

12. A system as claimed in claim 10 wherein electric power applied to the PCP device through said power ports bypasses said battery, thereby extending the life thereof.

13. A system as claimed in claim 10 wherein said station I/O port is connected to a processor for receiving and utilizing information from said at least one monitor.

14. A system as claimed in claim 13 wherein a source of electrical power and said processor are remote from said station and are connected thereto by a suitable link.

15. A system as claimed in claim 13 wherein said station includes said processor, an output mechanism and an input mechanism, said processor receiving information from said PCP device, controlling outputs on said output mechanism, and providing inputs to control said at least one monitor under control at least in part of said input mechanism.

16. A system as claimed in claim 10 wherein said slot includes a latch which engages when said PCP device is fully seated in said slot.

17. A system as claimed in claim 16 wherein said latch is a soft latch, providing some resistance to removal of the PCP device from the slot, but not significantly inhibiting such removal.

18. A system as claimed in claim 10 wherein said I/O ports are both optical ports.

19. A system as claimed in claim 10 wherein said power ports are one of galvanic connections, inductive couplings and RF connections.

20. A system as claimed in claim 10 wherein said station includes a battery for operating said station at least when said station has a PCP device mounted in its slot and is being moved, whereby said station is portable.
